(19) United States
(12) Patent Application Publication
(10) Pub. No.: US 2009/0247953 A1
(43) Pub. Date: Oct. 1, 2009

(54) MICRONEEDLE ADAPTOR FOR DOSED DRUG DELIVERY DEVICES

(75) Inventors: Yehoshua Yeshurun, Haifa (IL); Yotam Levin, Nes Ziona (IL); Meir Hefetz, Haifa (IL); Yoel Sefi, Merom Hagalil (IL); Gilad Lavi, Rishon Lezion (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NanoPass Technologies Ltd., Nes Ziona (IL)

(21) Appl. No.: 12/096,028

(22) PCT Filed: Dec. 10, 2006
(86) PCT No.: PCT/IL2006/001414

§ 371 Date: Oct. 7, 2008

(30) Foreign Application Priority Data
Dec. 8, 2005 (US) 60748280

(51) Int. Cl. A61M 5/32

(52) U.S. Cl. 604/173

(57) ABSTRACT

An adapter for achieving intradermal dosed delivery of a liquid by use of a dosed drug delivery device including a reservoir having a pierceable septum, the adapter having a connector including an attachment configuration for attachment to the dosed drug delivery device and a hollow needle deployed for piercing the septum. A liquid delivery interface, linked to the connector, includes a straight skin contact edge and one or more hollow microneedle adjacent thereto. The microneedle projects away from the skin contact edge. A flow path arrangement interconnects the needle and the at least one hollow microneedle.

### FIELD AND BACKGROUND OF THE INVENTION

The present invention relates to drug deliver devices and, in particular, it concerns a microneedle adapter for use with dosed drug delivery devices.

Dosed drug delivery devices, often referred to as “pen injectors,” are commonly used by diabetics for routine injection of insulin. Similar devices are also used for the delivery of hormones. Dosed drug delivery devices are a preferred means of delivery wherever the volume of drug delivered needs to be variable but accurate, small and frequently administered. Use of the term “pen injector” probably stems from the elongated pen-like form of many of the commercially available devices. However, unless otherwise specified, the term “pen injector” will be used herein interchangeably with the term “dosed drug delivery device” to refer generically to any and all free-standing portable device containing a plurality of doses of a therapeutic liquid which can be operated by a patient for self-injection to deliver metered doses of the liquid to the patient's body on a plurality of occasions. There are various kinds of pen injectors which may be variously classified according to different structural or functional features, such as: devices employing replaceable cartridges and devices which are disposed of when the contents are finished; devices with fixed dosage units or with various dialing and dosing features; devices with different flow activation mechanisms, ergonomics and design, reservoir systems and volume requirements etc.

Pen injectors are used with dedicated replaceable needle assemblies, referred to herein for convenience as “pen needles”. Commercially available pen needles known to the inventors all target the subcutaneous (SC) fatty layer and make use of tubular metal components (hypodermic stainless steel needles). Commercially available pen needles typically have lengths ranging from 1 mm to 25 mm.

Pen needles are configured to satisfy several requirements unique to pen injectors. On one side, they feature a connector for reversibly connecting to a liquid reservoir within the pen injector. The connector typically includes a hollow needle deployed for piercing a septum (resilient self-sealing membrane) integrated with the liquid cartridge, and an attachment configuration such as a threaded collar for attachment to the pen injector. On the other side, the pen needle features the skin-penetrating needle. The septum-piercing needle and the skin-penetrating needle are typically implemented as opposite ends of a single double-ended needle. The two ends typically have different point shapes, with the rear end configured to avoid coring of a hole in the septum and the front end shaped to minimize pain on penetration through the skin. This renders the double ended needle complex to manufacture. On the other hand, since a single continuous needle is used, there is typically no requirement of sealing between the needle and the surrounding connector body, often allowing the structure to be assembled without the sealing glue required for other hypodermic applications, and the “dead volume” of the needle is very small. For all of the above reasons, design considerations for pen needles are significantly different from those of other hypodermic needles, and such needles have attained a distinct status in the art, often being produced by specialist companies which deal exclusively with pen needles and other pen injector related accessories.

Miniature needles used for pen injectors typically project a minimum of 1 millimeter. In the case of a miniature needle of conventional hypodermic type (i.e., a metal tube formed with a beveled end), the bevel of the needle tip itself typically has a length of at least 0.8 mm, making it impossible to achieve sealed fluid delivery to penetration depths less than 1 mm.

In some published documents, it has been proposed to use “microneedles” as a delivery interface for pen injectors. For the purpose of the present description and claims, the term “microneedle” in its broadest sense is used to refer to a projecting structure with a projecting length of less than 1 millimeter. Examples of such documents include US patent application publication nos. US 2003/0050602 to Pettis and US 2003/0181863 to Ackley et al. Theoretically, application of microneedles to pen injectors promises various advantages attributed to intradermal delivery including, but not limited to, altered kinetics (depending on the formulation and the exact injection site, either accelerated absorption, such as may be beneficial for insulin delivery or delayed absorption, for example if a slow release formulation is used), improved response (for example intradermal delivery of vaccines may enhance immune response, allow for smaller doses, potentially lesser booster shots, better vaccination, etc), reduced trauma (since microneedles are smaller than conventional hypodermic needles), and minimally painful or painless injections. The last feature, in particular, is considered highly significant, possibly increasing patient compliance, improving quality of life, improving disease control and reducing expenses on treatment of disease complications. This is particularly relevant in the case of insulin injections for treatment of diabetes due to the direct relation between long term control of blood glucose levels and the prevalence of long term complications.

In practice, implementations of microneedles for pen injectors are not straightforward due to a number of practical problems. A first major problem of many microneedle designs relates to mechanical weakness of the microneedles which tend to fracture on contact with the skin, particularly when exposed to shear forces due to lateral movement. A further problem is that the highly elastic skin barrier tends to deform around the microneedles without the microneedles penetrating through the stratum corneum (SC). An additional problem is that of leakage around the microneedles' point of insertion and/or ejection of the needles by back-pressure generated during injection. Many designs are also prone to blockage of the bores of hollow microneedles due to punching-out of a plug of tissue during insertion through the skin.

Solutions to the aforementioned problems have been suggested in the context of applications such as infusion sets and syringes. Particularly, reference is made to a particularly advantageous robust microneedle structure as taught by U.S. Pat. No. 6,533,949, and to various microneedle insertion techniques as taught by PCT Patent Application Publication Nos. WO 03/074102 A2 and WO 2005/049107 A2, and in US Patent Application Publication No. US 2005/0209566 A1, these four publications mentioned in this paragraph all being hereby incorporated by reference in their entirety. However, these solutions have not previously been adapted to address the particular requirements of pen injectors. Furthermore, given the unique design considerations for pen needles, and the distinct status of pen needles as established in the art, such adaptations are not readily apparent to a person having ordinary skill in the art.

There is therefore a need for an adapter for achieving intradermal dosed delivery of a liquid by use of a dosed drug delivery device.

### SUMMARY OF THE INVENTION

The present invention is an adapter for achieving intradermal dosed delivery of a liquid by use of a dosed drug delivery device.

According to the teachings of the present invention there is provided, an adapter for achieving intradermal dosed delivery of a liquid by use of a dosed drug delivery device, the dosed drug delivery device including a reservoir having a pierceable septum, the adapter comprising: (a) a connector including an attachment configuration for attachment to the dosed drug delivery device and a hollow needle deployed for piercing the septum; (b) a liquid delivery interface mechanically linked to the connector, the liquid delivery interface including a substantially straight skin contact edge and a linear array of hollow microneedles deployed substantially adjacent to, and arrayed substantially parallel to, the skin contact edge, the microneedles projecting away from the skin contact edge; and (c) a flow path arrangement interconnecting the needle and the array of hollow microneedles.

According to a further feature of the present invention, each of the microneedles has a height, and wherein a distance between the skin contact edge and each of the microneedles is no greater than the height of the microneedles.

According to a further feature of the present invention, the substantially straight skin contact edge is formed as an edge of a block of material, the block of material being integrally formed with at least part of the attachment configuration.

According to a further feature of the present invention, an extensional direction of the hollow needle of the connector defines a primary flow axis, and wherein each of the hollow microneedles includes a flow channel defining an injection direction, the injection direction being inclined relative to the primary flow axis by an angle of at least 20 degrees.

According to a further feature of the present invention, the injection direction is inclined relative to the primary flow axis by an angle of between 30 degrees and 150 degrees.

According to a further feature of the present invention, the injection direction is inclined relative to the primary flow axis by an angle of about 90 degrees.

According to a further feature of the present invention, the hollow microneedles are integrally formed with a substrate.

According to a further feature of the present invention, the substrate has a substantially planar surface, and wherein each of the microneedles is formed by at least one wall standing substantially upright from the substantially planar surface and an inclined surface intersecting with the at least one wall.

According to a further feature of the present invention, each of the microneedles has a flow channel passing through the substrate and intersecting with the inclined surface.

According to a further feature of the present invention, the microneedles are formed from silicon.

There is also provided according to the teachings of the present invention, a combination of the aforementioned adapter with a dosed drug delivery device, the combination further including a dosed drug delivery device having a liquid reservoir including a pierceable septum, the adapter being connected to the dosed drug delivery device so that the hollow needle pierces the septum thereby bringing the microneedles into flow connection with contents of the reservoir.

According to a further feature of the present invention, the reservoir contains a quantity of insulin. Alternatively, the reservoir contains a quantity of a fertility hormone. In a further alternative, the reservoir contains a quantity of a growth hormone. In yet a further alternative, the reservoir contains a quantity of a vaccine.

There is also provided according to the teachings of the present invention, an adapter for achieving intradermal dosed delivery of a liquid by use of a dosed drug delivery device, the dosed drug delivery device including a reservoir having a pierceable septum, the adapter comprising: (a) a connector including an attachment configuration for attachment to the dosed drug delivery device and a hollow needle deployed for piercing the septum; (b) a liquid delivery interface mechanically linked to the connector, the liquid delivery interface including a substantially straight skin contact edge and at least one hollow microneedle deployed substantially adjacent to the skin contact edge, the at least one microneedle projecting away from the skin contact edge; and (c) a flow path arrangement interconnecting the needle and the at least one hollow microneedle.

### BRIEF DESCRIPTION OF THE DRAWINGS

The invention is herein described, by way of example only, with reference to the accompanying drawings, wherein:

FIG. 1 is an isometric view of a preferred form of a linear array of microneedles for use in the adapters of the present invention;

FIG. 2A is a exploded isometric view of a first embodiment of an adapter, constructed and operative according to the teachings of the present invention, for use with a dosed drug delivery device to achieve intradermal dosed delivery of a liquid;

FIG. 2B is a isometric partially-cut-away view of the adapter of FIG. 2A as assembled prior to use;

FIG. 2C is a cross-sectional view taken through the adapter of FIG. 2B after removal of protective covers;

FIG. 3 is a cross-sectional view similar to FIG. 2C taken through a second embodiment of an adapter, constructed and operative according to the teachings of the present invention, for use with a dosed drug delivery device to achieve intradermal dosed delivery of a liquid;

FIG. 4A is a side view of the adapter of FIG. 2B assembled on a pen injector ready for use;

FIG. 4B is an enlarged view of the region of FIG. 4A including the adapter;

FIG. 5A is a side view of the adapter of FIG. 3 assembled on a pen injector ready for use;

FIG. 5B is an enlarged view of the region of FIG. 5A including the adapter;

FIG. 6A is a view similar to FIG. 4B after interfacing of the adapter with the skin of a user;

FIG. 6B is a view similar to FIG. 6A after injection of a dose of the liquid;

FIG. 7A is a view similar to FIG. 5B after interfacing of the adapter with the skin of a user; and

FIG. 7B is a view similar to FIG. 7A after injection of a dose of the liquid.

### DESCRIPTION OF THE PREFERRED EMBODIMENTS

The present invention is an adapter for use with a dosed drug delivery device to achieve intradermal dosed delivery of a liquid.

The principles and operation of adapters according to the present invention may be better understood with reference to the drawings and the accompanying description.

By way of introduction, the present invention relates to an adaptation of a microneedle drug delivery interface and corresponding technique described in US Patent Application Publication No. US 2005/0209566 A1 to render it suitable for use as a disposable drug delivery interface for pen injectors. The adapter most preferably employs microneedles produced by MEMS techniques from a single-crystal block of material such as silicon according to the teachings of U.S. Pat. No. 6,533,949. Alternatively, various other forms of microneedles and/or other materials may be used, such as are taught in U.S. Pat. No. 6,503,231 to Prausnitz et al. These documents are hereby incorporated by reference herein and provide helpful background to the present invention.

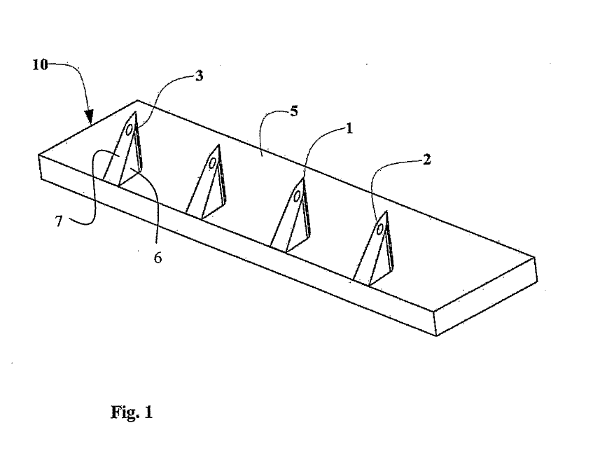

Referring now to the drawings, FIG. 1 shows a particularly preferred implementation of a linear array 10 of microneedles for use in the adapter of the present invention. Specifically, linear array 10 includes a number of hollow microneedles, typically between 1 and 10, more preferably between 3 and 6, and in the preferred case illustrated here, 4. Each microneedles has a penetrating point 1, a liquid flow channel 2 and preferably also a cutting edge 3. The microneedles are preferably integrally formed with a substrate 5, having a substantially planar surface. In the preferred implementation shown here, each microneedle is formed by a set of one or more walls 6 standing substantially upright from the substantially planar surface of substrate 5, and an inclined surface 7 intersecting with walls 6. Flow channel 2 is preferably formed as a bore passing through substrate 5 and intersecting with inclined surface 7. The linear microneedle array 10 is preferably formed using a combination of dry etching and wet etching processes from a single crystal of material, most preferably silicon, by techniques such as those described in detail in the aforementioned U.S. Pat. No. 6,533,949. Preferred dimensions for the microneedles for this application are a total height in the range of 250 to 650 microns, and most preferably 450±30 micron. The flow channel 2 may be round or of other cross-sectional shape, and preferably has a minimum internal diameter of about 45±10 microns if round, and an equivalent minimum cross-sectional area if otherwise shaped.

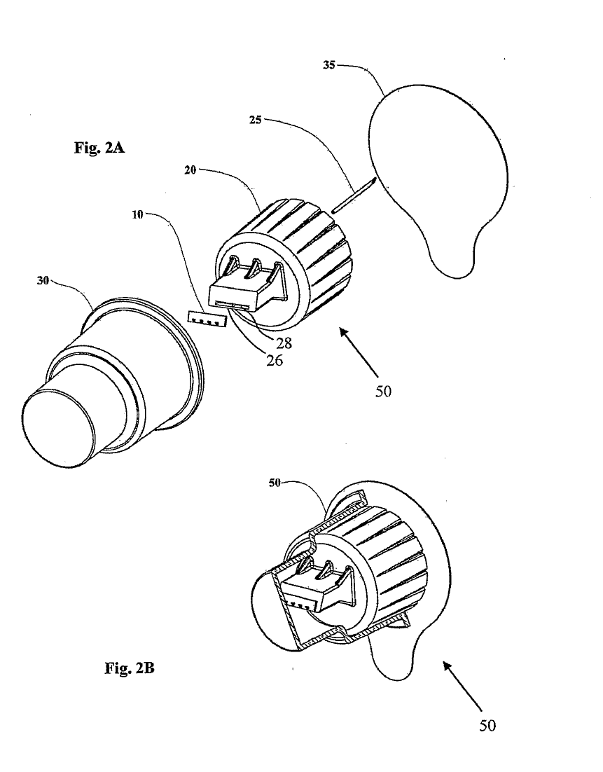

Turning now to FIGS. 2A-2C, these illustrate a first preferred embodiment of an adapter, generally designated 50, constructed and operative according to the teachings of the present invention, for achieving intradermal dosed delivery of a liquid by use of a dosed drug delivery device. Generally speaking, adapter 50 includes a connector including an attachment configuration 22 for attachment to a dosed drug delivery device, and a hollow needle 25 deployed for piercing a septum of a reservoir (typically, a cartridge such as a shell vial with moveable plug) of the dosed drug delivery device. Adapter 50 also features a liquid delivery interface 24, mechanically linked to the connector, including a substantially straight skin contact edge 26 and linear array 10 of hollow microneedles deployed substantially adjacent to, and arrayed substantially parallel to, skin contact edge 26. The microneedles preferably projecting away from the skin contact edge 26. A flow path arrangement 28 interconnects needle 25 with the flow channels of the microneedles in linear array 10.

Prior to use, adapter 50 is preferably protected by a front cover 30, as shown in FIGS. 2A and 2B, which protects the microneedles from accidental mechanical damage. Front cover 30 preferably also seals against a peel-off backing sheet 35 which prevents accidental contact with needle 25 and maintains sterility, together forming a pre-sealed sterile packaging for adapter 50. The device can be sterilized using common methods such as Gamma irradiation or exposure to Ethylene Oxide.

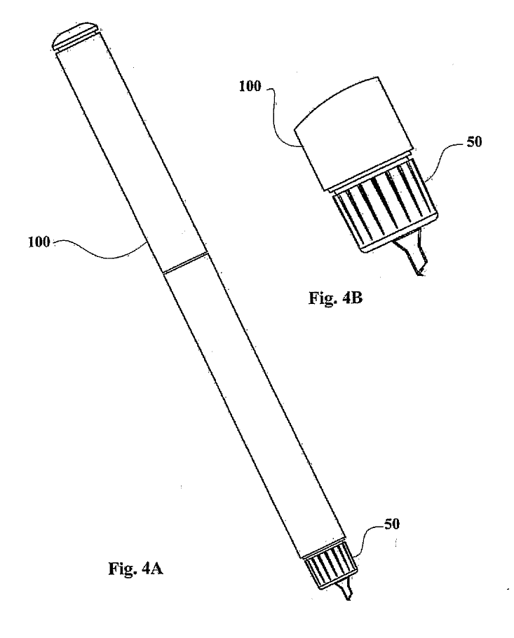

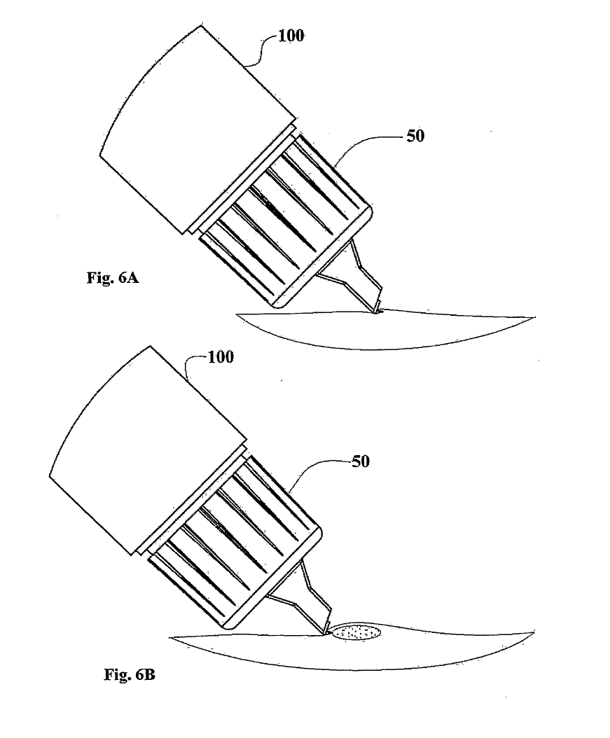

The mode of use of adapter 50 will be understood with reference to FIGS. 4A, 4B, 6A and 6B. First, after removal of backing sheet 35, attachment configuration 22 is attached instead of a pen needle to a conventional pen injector 100, as shown in FIG. 4A and enlarged in FIG. 4B. As adapter 50 is attached to the pen injector, needle 25 (not visible in this view) pierces the septum of the liquid vial or cartridge within the pen injector, thereby forming a flow connection to the microneedles. Then, as shown in FIG. 6A, the assembled device is brought into contact with the user's skin and pushed gently with a vector of motion having a non-zero component parallel to the initial surface of the skin (to the right as shown) so as to achieve penetration with the microneedles projecting primarily sideways, parallel to the initial surface of the skin. This form of insertion achieves numerous advantages over conventional perpendicular insertion, as detailed in the aforementioned US Patent Application Publication No. US 2005/0209566 A1. The pen injector is then actuated in the normal manner to achieve injection of the desired dose of the contained liquid, as illustrated schematically in FIG. 6B.

At this stage, it will already be apparent that the adapter of the present invention provides profound advantages over the prior art. Specifically, all pen injector art known to the inventors maintains the conventional approach of perpendicular insertion of the needle(s) into the skin, thereby suffering from the aforementioned limitations of penetration depths in excess of 1 millimeter for conventional needles, or problems of incomplete penetration and ejection by back pressure for microneedles. In contrast, by providing the unique geometry of the present invention in which an array of microneedles are adjacent to a skin contact edge, the present invention facilitates insertion of microneedles so that the microneedle flow channels are directed sideways, i.e., at an angle in the range of ±30° from the initial plane of the skin surface, into tissue not squashed under the device. As a result, the adapter of the present invention allows a pen injector to be used to achieve shallower intradermal liquid delivery than is possible using conventional devices, and is believed to encounter reduced flow impedance and achieve better intradermal distribution than would otherwise be achieved. These and other advantages of the present invention will be better understood with reference to the following description.

Turning now to the features of the present invention in more detail, skin contact edge 26 is preferably formed as an edge of a block of material which supports the microneedle array 10. Most preferably, this block is integrally formed with at least part of the attachment configuration. Thus, in the example of FIGS. 2A-2C, adapter 50 is most preferably formed from a combination of only three elements: microneedle array 10, needle 25, and a unitary body 20 formed from molded polymer material which provides both the attachment configuration (in this case, a threaded collar) and support for microneedle array 10. Most preferably, body 20 is formed from molded polycarbonate. This three-element implementation minimizes production costs, rendering the adapter suitable for disposable use as a pen needle substitute.

Body 20 also preferably defines any flow paths 28 required to interconnect needle 25 with the flow channels of the microneedles. In the preferred implementation shown, this includes a transverse open channel formed under the point of attachment of microneedles array 10 so that, when the substrate is attached by use of adhesive, welding or other known methods, the channel together with the rear surface of the substrate forms a closed channel for distributing liquid from needle 25 to all of the microneedles. The positioning of this channel is chosen to intersect a central axis of the adapter 50 along which needle 25 is aligned, thereby simplifying manufacture of body 20, as will be clear to one familiar with plastic injection molding technology.

The form of body 20 is chosen to facilitate bringing the microneedles into contact with the skin in the correct orientation. In the preferred example shown here, body 20 is formed with a forward projecting portion which is roughly rectangular in cross-section, having a major dimension parallel to the extensional direction of microneedle array 10 and a minor dimension perpendicular thereto. The microneedles are preferably deployed with the inclined surface having flow channel 2 facing downwards, i.e., inwards towards the depth of the tissue.

In order to optimize the sideways insertion geometry, the microneedles are preferably close to edge 26. Preferably, a distance between skin contact edge 26 and each of the microneedles, defined as the distance between edge 26 and the closest part of the base of the microneedles, is no greater than the height of the microneedles themselves as measured perpendicular to the surface of the substrate. Most preferably, the microneedles are juxtaposed with their base starting substantially at edge 26. Parenthetically, it should be noted that edge 26 itself may be provided by either the edge of the substrate of microneedle array 10 or by an edge of body 20 adjacent to the array 10.

It will be noted that adapter 50 causes a significant deflection of the flow direction between the axial direction of the dosed drug delivery device (corresponding to the direction of needle 25) and the injection direction as defined by the flow channels of the microneedles. This deflection is preferably at least about 20 degrees and, more preferably, between about 30 and about 150 degrees. In the case shown here, the deflection is roughly 40 degrees. Nevertheless, in order to achieve an injection direction near parallel to the initial plane of the skin, this embodiment requires deployment of the pen injector at an inclination as shown in FIGS. 4A, 4B, 6A and 6B.

Parenthetically, although the device is illustrated here in a preferred embodiment in which a linear array of microneedles is used, it should be noted that a minimal embodiment in which a single microneedle is used in proximity to skin contact edge 26 also falls within the broad scope of the present invention.

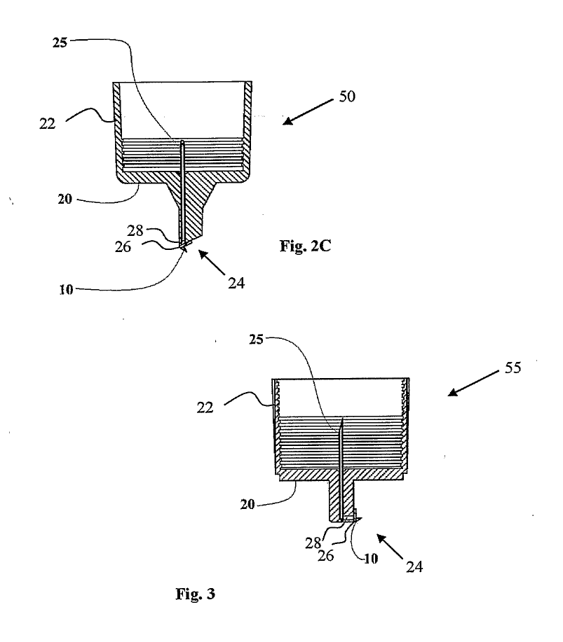

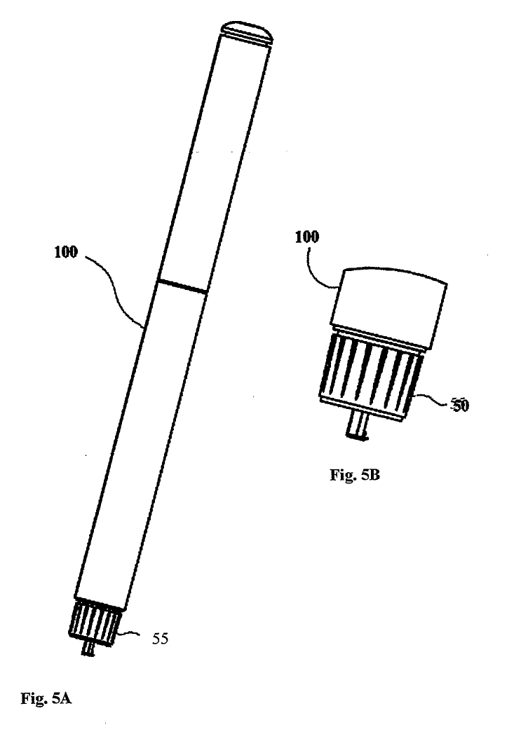

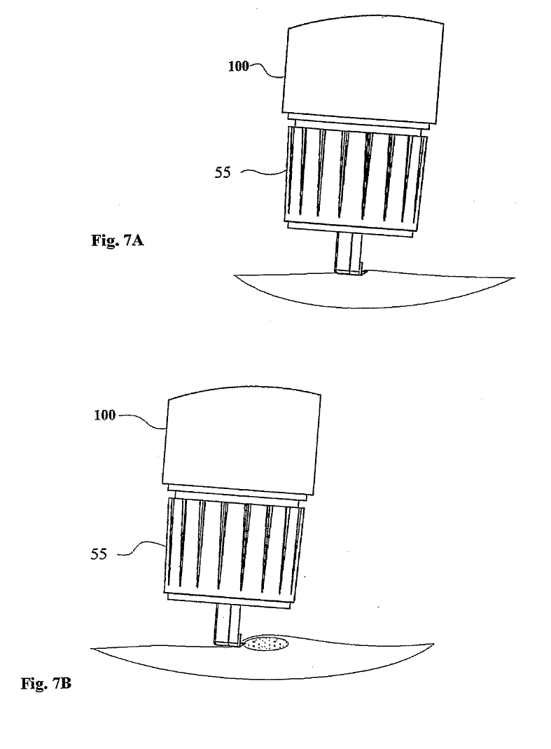

FIGS. 3, 5A, 5B, 7A and 7B illustrate an alternative embodiment of an adapter, generally designated 55, which provides a larger deflection of the flow direction, namely, about 90 degrees. This orientation achieves sideways injection while allowing the device to be held generally orthogonally to the initial skin surface, in a manner more similar to the orientation of a pen injector used with a conventional pen needle. Initial insertion of the microneedles into the skin surface is preferably performed at a slight angle, as illustrated in FIGS. 5A and 5B, and typically requires a slight turning motion, applying an anticlockwise turning moment in the orientation as illustrated in FIG. 7A.

In other respects, the structural features and function of adapter 55 will be understood by analogy to the corresponding features and function of adapter 50 described above, with like elements being labeled similarly.

It will be appreciated that the present invention may be used to advantage in a large number of drug delivery applications, including both applications for which pen injectors are conventionally used and new applications for which the shallow intradermal delivery achieved by the present invention may be advantageous. Examples of applications include, but are not limited to, administering: insulin, fertility hormones, growth hormone and vaccines. Other applications include, but are not limited to, the substances and modes of treatment mentioned in US Patent Application Publication No. 2005/0163711 A1, which is hereby incorporated by reference herein.

It will be appreciated that the above descriptions are intended only to serve as examples, and that many other embodiments are possible within the scope of the present invention as defined in the appended claims.

What is claimed is:

1. An adapter for achieving intradermal dosed delivery of a liquid by use of a dosed drug delivery device, the dosed drug delivery device including a reservoir having a pierceable septum, the adapter comprising:
(a) a connector including an attachment configuration for attachment to the dosed drug delivery device and a hollow needle deployed for piercing the septum;
(b) a liquid delivery interface mechanically linked to said connector, said liquid delivery interface including a substantially straight skin contact edge and a linear array of hollow microneedles deployed substantially adjacent to, and arrayed substantially parallel to, said skin contact edge, said microneedles projecting away from said skin contact edge; and
(c) a flow path arrangement interconnecting said needle and said array of hollow microneedles.

2. The adapter of claim 1, wherein each of said microneedles has a height, and wherein a distance between said skin contact edge and each of said microneedles is no greater than said height of said microneedles.

3. The adapter of claim 1, wherein said substantially straight skin contact edge is formed as an edge of a block of material, said block of material being integrally formed with at least part of said attachment configuration.

4. The adapter of claim 1, wherein an extensional direction of said hollow needle of said connector defines a primary flow axis, and wherein each of said hollow microneedles includes a flow channel defining an injection direction, said injection direction being inclined relative to said primary flow axis by an angle of at least 20 degrees.

5. The adapter of claim 4, wherein said injection direction is inclined relative to said primary flow axis by an angle of between 30 degrees and 150 degrees.

6. The adapter of claim 4, wherein said injection direction is inclined relative to said primary flow axis by an angle of about 90 degrees.

7. The adapter of claim 1, wherein said hollow microneedles are integrally formed with a substrate.

8. The adapter of claim 7, wherein said substrate has a substantially planar surface, and wherein each of said microneedles is formed by at least one wall standing substantially upright from said substantially planar surface and an inclined surface intersecting with said at least one wall.

9. The adapter of claim 8, wherein each of said microneedles has a flow channel passing through said substrate and intersecting with said inclined surface.

10. The adapter of claim 7, wherein said microneedles are formed from silicon.

11. A combination of the adapter of claim 1 with a dosed drug delivery device, the combination further including a dosed drug delivery device having a liquid reservoir including a pierceable septum, the adapter being connected to said dosed drug delivery device so that said hollow needle pierces said septum thereby bringing said microneedles into flow connection with contents of said reservoir.

12. The combination of claim 11, wherein said reservoir contains a quantity of insulin.

13. The combination of claim 11, wherein said reservoir contains a quantity of a fertility hormone.

14. The combination of claim 11, wherein said reservoir contains a quantity of a growth hormone.

15. The combination of claim 11, wherein said reservoir contains a quantity of a vaccine.

16. An adapter for achieving intradermal dosed delivery of a liquid by use of a dosed drug delivery device, the dosed drug delivery device including a reservoir having a pierceable septum, the adapter comprising:
(a) a connector including an attachment configuration for attachment to the dosed drug delivery device and a hollow needle deployed for piercing the septum;
(b) a liquid delivery interface mechanically linked to said connector, said liquid delivery interface including a substantially straight skin contact edge and at least one hollow microneedle deployed substantially adjacent to said skin contact edge, said at least one microneedle projecting away from said skin contact edge; and
(c) a flow path arrangement interconnecting said needle and said at least one hollow microneedle.

17. The adapter of claim 16, wherein said at least one hollow microneedle is implemented as a plurality of hollow microneedles deployed in a substantially linear array substantially parallel to said skin contact edge.
